(19) United States
(12) Patent Application Publication
(10) Pub. No.: US 2011/0177503 A1
(43) Pub. Date: Jul. 21, 2011

(54) CARDIAC RISK STRATIFICATION BY NOS1AP GENOTYPING

(76) Inventors: Peter M. Spooner, Baltimore, MD (US); Silvia G. Priori, Pavia (IT); Carlo Napolitano, Pavia (IT)

(21) Appl. No.: 12/974,787

(22) Filed: Dec. 21, 2010

Related U.S. Application Data
(60) Provisional application No. 61/296,588, filed on Jan. 20, 2010.

(51) Int. Cl. C12Q 1/68; A61B 5/0402; C40B 30/04

(52) U.S. Cl. 435/6.11; 600/509; 506/9

(57) ABSTRACT

A risk-conferring genetic modifier is found in a large LQTS cohort. A NOS1AP tag SNP genotype provides an additional clinical assessment, which helps assess risk and choice of therapeutic strategies in LQTS as well as other conditions such as Brugada Syndrome, and catecholaminergic polymorphic ventricular tachycardia (CPTV).

### TECHNICAL FIELD OF THE INVENTION

This invention is related to the area of heart disease. In particular, it relates to prediction of risk of adverse cardiac events.

### BACKGROUND OF THE INVENTION

LQTS is a genetic disorder caused by mutations that affect genes encoding ion-channel subunits or proteins that indirectly modulate the function of ion channels. The disease derives its name from the diagnostic electrocardiographic feature of prolonged repolarization (QT interval). The heart of an LQTS patient is predisposed to develop life-threatening arrhythmias, often when exposed to physical or psychological stress (1). Beta-blockers are the mainstay therapy in LQTS (1). The use of the implantable cardio-defibrillator (ICD) is recommended in patients at high risk of sudden cardiac death and in those who continue to have cardiac events despite anti-adrenergic therapy (2). The identification of markers of increased arrhythmic risk is a major goal of epidemiological research in LQTS. In addition to extent of QT prolongation, which is the major prognostic indicator (3), gender and genetic locus have also proven useful in predicting adverse events (4,5). More recently the effort to account for the large phenotypic variability of the disease (6) has focused on the search for additional genetic modifiers. Polymorphisms at additional loci in LQTS genes have been proposed as factors that may modulate QT interval (7); however, conclusive evidence that one or more polymorphisms influence the phenotype, including risk of cardiac events of the disease has been lacking.

Prevalence of clinical LQTS in the general population was recently estimated at ˜1:2500 (Crotti, et al 2009) and a large body of work has shown that across large populations of LQTS patients the disease may be caused by hundreds of different mutations in at least ten genes. The cause of 20-35% of such cases however still remains undiscovered. Molecular diagnosis of many forms of LQTs can often be determined by DNA sequencing of coding regions in exons of cardiac Na and K channel genes, and this service is available commercially in the United States from at least two separate companies, which collectively have genotyped several thousand individuals. Positive molecular identification of a causal mutation in the initially presenting proband or in first, second, and third degree relatives is used to aid the risk stratification of affected individuals. Equally important, identification of a causal mutation is used to guide therapy, specifically to identify subjects and family members at high risk who may require life-long therapy with an implantable cardio-defibrillator (ICD). LQTS genotyping has also been used as a form of molecular autopsy in cases of unexplained death, principally in infants and young children.

Recently, a genome-wide association study identified an association between SNPs in the NOS1AP gene and QT interval (8). The influence of NOS1AP SNPs on QT was subsequently replicated in follow-up studies in the Framingham Heart and Rotterdam populations (8-10) as well as in other cohorts (11-16). Recently, the role of NOS1AP gene variants in sudden cardiac death has been reported in the general population (17). There is a continuing need in the art to identify and treat high risk cardiac patients.

### SUMMARY OF THE INVENTION

According to one aspect of the invention a method is provided for stratifying female patients for risk of adverse cardiac events, syncope, Torsade de pointes, or cardiac arrest. The patients have a syndrome selected from the group consisting of LQTS1-10, short QT syndrome, Brugada Syndrome, catecholaminergic polymorphic ventricular tachycardia (CPTV) and cryptic, familial QT abnormality, but the patients do not have a KCNQ1:A341V mutation. Genomic sequences of a female patient are tested for the presence of a minor allele of NOS1AP. The minor allele may be identified with tag SNPs rs10494366, rs54657139, rs16847548, or a marker in positive linkage disequilibrium with at least one of said minor alleles. The electrographic QT interval in the female patient is tested. The patient is identified as having a higher risk if the patient has (a) at least one of said minor alleles or at least one of said markers in positive linkage disequilibrium, and (b) a QT prolongation, relative to the average for a population of females with said syndrome who do not have both a QT prolongation and one of the minor alleles.

According to another aspect of the invention a method is provided for stratifying male patients for risk of adverse cardiac events, syncope, Torsade de pointes, or cardiac arrest. The patients have a syndrome selected from the group consisting of LQTS1-10, short QT syndrome, Brugada Syndrome, catecholaminergic polymorphic ventricular tachycardia (CPTV) and cryptic, familial QT abnormality, but the patients do not have a KCNQ1:A341V mutation. Genomic sequences of a male patient are tested for the presence of a minor allele of NOS1AP. The minor allele may be identified with tag SNPs rs10494366, rs54657139, rs16847548, or a marker in positive linkage disequilibrium with at least one of said minor alleles. The patient is identified as having a higher risk than the average for a population of male patients that have said syndrome if the patient has one of the minor alleles or a marker in positive linkage disequilibrium with the minor allele.

Yet another aspect of the invention provides a method of stratifying female patients for risk of adverse cardiac events, syncope, Torsade de pointes, or cardiac arrest. The patients have a prolonged QT interval and have a syndrome selected from the group consisting of LQTS1-10, short QT syndrome, Brugada Syndrome, catecholaminergic polymorphic ventricular tachycardia (CPTV) and cryptic, familial QT abnormality. A minor allele of NOS1AP which may be identified with tag SNPs rs10494366, rs54657139, rs16847548, or a marker in linkage disequilibrium with rs10494366, rs54657139, or rs16847548 is determined in genomic sequences of one of the patients. The patient is identified as having a higher risk than the average for a female population having prolonged QT and said syndrome.

Still another aspect of the invention is a method of stratifying female patients for risk of adverse cardiac events, syncope, Torsade de pointes, or cardiac arrest. The patients' genomic sequences have a minor allele of NOS1AP which may be identified with tag SNPs at rs10494366, rs54657139, rs16847548, or a marker in linkage disequilibrium with rs10494366, rs54657139, or rs16847548. The patients have a syndrome selected from the group consisting of LQTS1-10, short QT syndrome, Brugada Syndrome, catecholaminergic polymorphic ventricular tachycardia (CPTV) and cryptic, familial QT abnormality. A prolonged QT interval is determined in one of the female patients. The patient is identified as having a higher risk than the average for a population having the minor allele of NOS1AP which may be identified with tag SNPs at rs10494366, rs54657139, rs16847548, or a marker in linkage disequilibrium with rs10494366, rs54657139, or rs16847548.

An additional aspect of the invention is a method of stratifying patients for risk of adverse cardiac events, syncope, Torsade de pointes, and cardiac arrest. A minor allele of NOS1AP which may be identified with tag SNPs at rs54657139, rs16847548, or a marker in linkage disequilibrium with rs54657139, or rs16847548 is determined in genomic sequences of a patient. The patient is taking or may be prescribed a drug that prolongs QT. The patient is identified as having a higher risk of adverse cardiac events and cardiac arrest than the average for a population taking the drug that does not have said minor allele.

These and other embodiments which will be apparent to those of skill in the art upon reading the specification, provide the art with additional methods of assessing risk of adverse cardiac events.

### BRIEF DESCRIPTION OF THE DRAWINGS

FIG. 1. Survival according to QTc and gender. Kaplan-Meier survival analysis showing the cumulative incidence of cardiac events according to gender and QTc interval duration in LQTS population (n=901). Females with QTc≧500 ms (black continuous line) demonstrate the worst outcome.

FIG. 2. Effect of rs10494366 minor allele in LQTS with QTc<500 ms—Cox regression analysis showing the independent effect of NOS1AP rs10494366 on cumulative events-free survival in LQTS population with QTc<500 ms. The presence of at least one rs10494366 minor allele confers a 1.63 fold increased risk of cardiac events as compared with major allele homozygotes.

FIG. 3. LQTS risk stratification scheme—Combined Hazard Ratios (cHR) from Cox Regression model identifies risk categories in the LQTS population. Each box shows the cHR for patients with different clinical and genetic profile. To calculate the cHR we multiply the hazard ratio of each independent predictor of events derived from Cox multivariate analysis with the following reference category (HR=1) the following features: LQT1, males, QT<500 ms homozygous for the common allele of NOS1AP rs10494366. Accordingly the cHR for the category “LQT1, Males, QTc≧500 ms, carriers of minor allele of NOS1AP rs10494366” is calculated as follows: LQT1 corresponds to HR=1, Male corresponds to HR=1; QTc≧500 ms corresponds to HR 2.78 and carrier of minor allele of NOS1AP rs10494366 correspond to HR=1.47. The cHR is therefore 1×1×2.78×1.47=4.08 as shown in the corresponding upper left corner orange box in the figure. Reference category is represented by Males LQT1 with QTc<500 ms homozygous for the rs10494366 common allele (“TT”).

FIG. 4 shows the effect of rs10494366 minor allele in LQTS combined with gender and QT stratification.

FIG. 5. (Online FIG. 1.) Kaplan-Meier survival analysis showing the cumulative incidence of cardiac events according to rs14847548 genotype. Categories are defined according to the dominant model chosen as the most appropriate in our cohort (see methods). When included in the Cox model with QTc and gender the HR for RS14847548 was: 1.14 (95% CI: 0.83-1.46)

FIG. 6. (Online FIG. 2.) Kaplan-Meier survival analysis showing the cumulative incidence of cardiac events according to rs4657139 genotype. Categories are defined according to the dominant model chosen as the most appropriate in our cohort (see methods). When included in the Cox model with QTc and gender the HR for rs4657139 was: 1.25 (95% CI: 0.92-1.69)

FIG. 7. (Online FIG. 3.) Kaplan-Meier survival analysis showing the cumulative incidence of cardiac events according to rs10494366 genotype. Categories are defined according to the dominant model chosen as the most appropriate in our cohort (see methods). When included in the Cox model with QTc and gender the HR for rs10494366 was: 1.32 (95% CI: 0.96-1.82)

FIG. 8. (Online FIG. 4.) Kaplan-Meier survival analysis showing the cumulative incidence of cardiac events according to rs4657139 genotype in patients with QTc<500 ms. Categories are defined according to the dominant model chosen as the most appropriate in our cohort (see methods). When included in the Cox model with QTc and gender the HR for rs4657139 was: 1.30 (95% CI: 0.88-1.93).

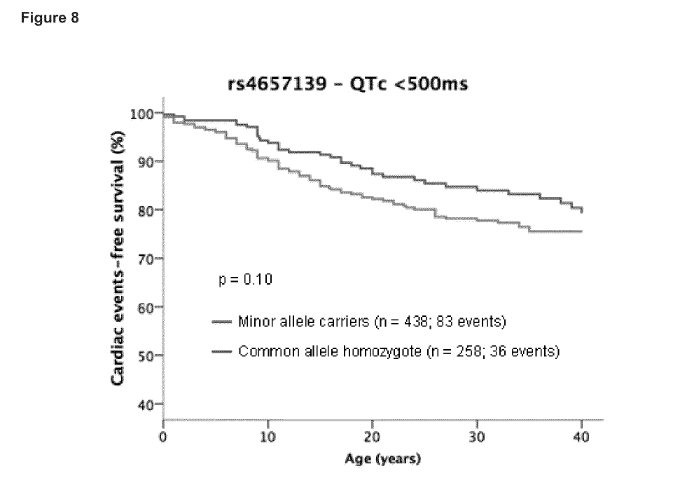

FIG. 9. (Online FIG. 5.) Kaplan-Meier survival analysis showing the cumulative incidence of cardiac events according to rs16847548 genotype in patients with QTc<500 ms. Categories are defined according to the dominant model chosen as the most appropriate in our cohort (see methods). When included in the Cox model with QTc and gender the HR for rs16847548 was: 1.26 (95% CI: 0.88-1.80)

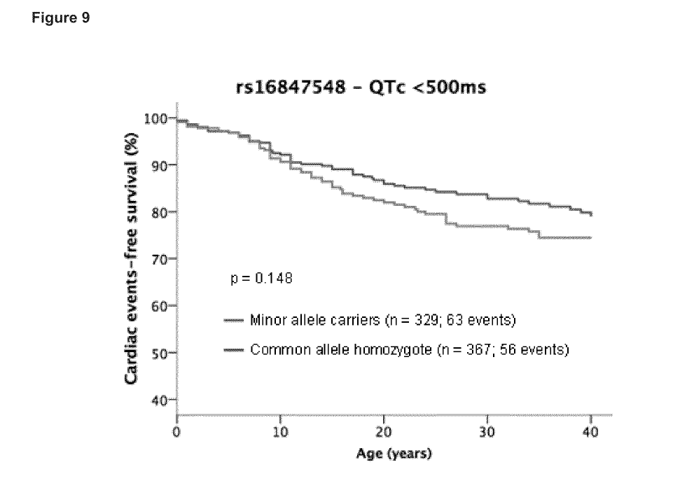

FIG. 10. (Online FIG. 6.) Kaplan-Meier survival analysis showing the cumulative incidence of cardiac events according to rs10494366 genotype in patients with QTc<500 ms. Categories are defined according to the dominant model chosen as the most appropriate in our cohort (see methods). When included in the Cox model with QTc and gender the HR for rs10494366 was: 1.63 (95% CI: 1.06-2.50—p=0.03)

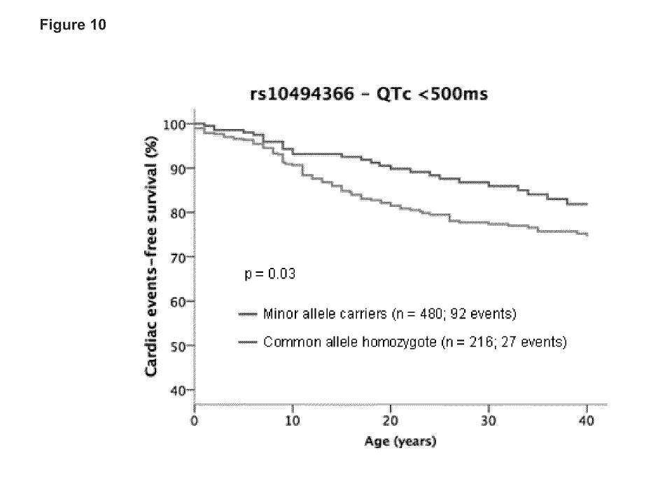

FIG. 11. (Online FIG. 7.) Kaplan-Meier survival analysis showing the cumulative incidence of cardiac arrests according to rs10494366 genotype in patients with QTc<500 ms. Categories are defined according to the dominant model chosen as the most appropriate in our cohort (see methods).

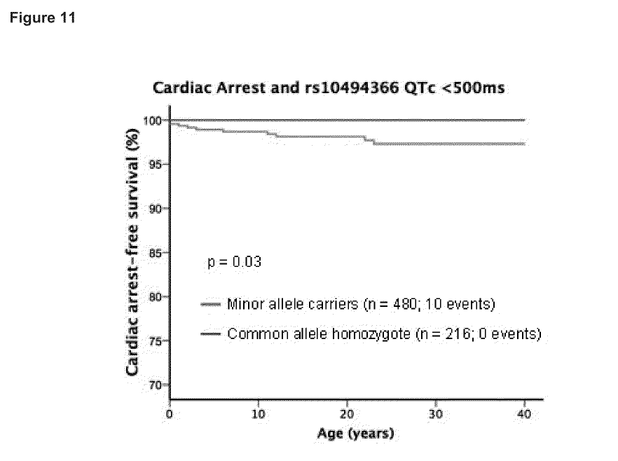

### DETAILED DESCRIPTION OF THE INVENTION

The inventors have found that NOS1AP genotyping is informative for LQTS patients, adding critical information to their clinical assessment. The data indicate that specific variants in NOS1AP non-coding sequences act via at least two distinct mechanisms that can be independently assessed. Regardless of the mechanism, however, in both cases, risk of adverse cardiac events is increased when the minor allele is present.

One pathway involves 5′ NOS1AP variants which extend the QT interval, reflecting prolongation of cardiac repolarizaton and concordant electrical instability due to alterations in the myocardial action potential. It is not known if this effect is the result of changes in the expression of NOS coding or non-coding RNA, or whether it involves changes in the synthesis or function of the encoded protein CAPON. This is similar to the QT prolonging effect of many of the primary ion channel mutations underlying LQTS itself. This pathway can be assessed using tag-SNPS such as rs16847548 and rs4657139 or additional tag-SNPs in linkage disequilibrium (LD) with these loci.

The second pathway is associated with the risk allele interrogated with SNP rs10494366, which does not appear to alter QT interval in these patients, but is in fact associated with a >60% decrease in event free survival in patients with LQT1, LQT2 and LQT3. Surprisingly, we found that this is a pathway of risk amplification, whereby the length of the QT interval is extended by ˜50% regardless its initial value. Finally we note that because of molecular and biological similarities of these different forms of the LQTS, similar or identical effects likely occur in patients with other “ion channelopathy” syndromes, where QT prolongation is an established mediator of arrhythmia and sudden cardiac death (SCD) risk. These include the inherited “Brugada Syndrome,” a cardiac arrhythmic disorder at least as prevalent as LQTS, and the less frequent condition of CPTV (catecholaminergic polymorphic ventricular tachycardia) associated with mutations in Ca² handling genes, such as RYR2. As with the LQTS conditions, there is a need to improve risk analysis through improved diagnosis and improved identification of those patients who will benefit from drug therapy (e.g., anti-beta adrenergics) and those who would benefit from implantation of an implantable cardioverter-defibrillator (ICD). These are clinical decisions with high medical and economic consequences.

Any of the three SNP markers identified in the examples below may be used to detect a minor allele of NOS1AP. Similarly, any marker that is in positive linkage disequilibrium with at least one of these markers can be used. Markers in high positive linkage disequilibrium rarely have a recombination event between them, due to proximity and other factors. SNP markers can be determined by any means known in the art. They can be determined, without limitation, by sequencing, by hybridization, by allele-specific amplification, by allele-specific extension, by interrogation on a probe chip. Probes or primers for testing may be attached to any solid support, such as a bead, or resin, or can be arrayed in wells or on a chip. Any method known in the art may be used.

The three markers described here are known in the art. These markers, rs10494366, rs16847548, and rs4657139 carry the meaning that they have in the scientific literature. Their location, sequence, and means of identifying them are known. These markers occur in the 5′ or the intronic regions of NOS1AP. The minor alleles may possibly affect expression of the CAPON-coding exons, or it may possibly affect expression of RNA molecules that are regulatory. The mechanism of action of these minor alleles is not fully elucidated.

Assessing the sex of an individual is performed as is conventional in the art. Any of the various indications of an individual's sex may be used. These may be genetic, hormonal, genital, secondary sexual characteristics, or other means. Again, the manner of determining can be any known in the art. The mechanism by which the combination of the individual's sex and the minor alleles affect the risk is not fully elucidated.

Stratification assigns individuals to groups with similar characteristics. The groups may be, for example, for testing or prescribing any of the following non-limiting treatments: drugs, surgery, diet, exercise, or other intervention.

QT prolongation can be determined according to any means known in the art. Typically this is determined by ECG measurements. The QT interval is a measure of the time between the start of the Q wave and the end of the T wave in the heart's electrical cycle. The QT interval is often corrected for heart rate. Abnormally prolonged or shortened QT, predisposes one to develop ventricular arrhythmias. There are many reasons for abnormal QT intervals, including genetic changes in ion channels, adverse drug reactions, pathological conditions, and hypercalcemia. Drugs such as haloperidol and quinidine can prolong the QT interval. Some antiarrhythmic drugs, like amiodarone or sotalol work by inducing a pharmacological QT prolongation. QTc of 440 or 450, for makes and females, respectively are often used as a risk cut-off value, but other cut-offs may be used in specific populations.

Some drugs such as haloperidol and methadone prolong the QT interval. Thus if a physician is considering prescribing one of these drugs, its interaction with the effect of the NOS1AP allele and/or native QT interval may be considered as a negative factor. These drugs include: amiodarone, arsenic trioxide, astemizole, bepridil, chloroquine, chlorpromazine, cisapride, clarithromycinm, disopyramide, dofetilide, domperidone, droperidol, erythromycin, halofantrine, haloperidol, ibutilide, levomethadyl, mesoridazine, pentamidine, pimozide, probucol, procainamide, quinidine, sotalol, sparfloxacin, terfenadine, and thioridazine.

Heritability of QT Interval

Dissecting how genetic variation alters the electrical substrate has been the target of many investigations and an area of intense current research. Identifying the genes that modulate QT interval and their effect on the occurrence of cardiac events has, accordingly, become one of the primary goals of molecular studies of cardiac excitability. Several groups have performed candidate gene or genome wide association studies (7,8,11,22-24) to identify genetic modifiers of repolarization. Candidate gene approach was initially used with the idea that variants of ion channels causing also the full-blown LQTS were potential modifiers. However observations based on this hypothesis have been either inconsistent or remained unconfirmed (7,23-25). More recently, data from genome wide association studies identified several variants of the NOS1AP gene that play a major role in the modulation of cardiac repolarization (8,26,27). The link between NOS1AP and QTc was provided first by Arking et al (8) who identified SNPs associated with QT duration in the upstream 5′ regulatory region of NOS1AP. Such association came as a surprise since this gene had not been previously associated with cardiac electrogenesis, although it had been implicated in schizophrenia and dystrophic muscular atrophy (28,29). NOS1AP encodes the CAPON protein, an adaptor molecule, which binds the neural nitric oxide synthase (Nos1) via a C-terminal PDZ domain, with scaffold and signaling ligands (e.g., dexras, dystrophins). Functional studies show that CAPON is a spatial controller of NO production and it modulates action potential duration via an effect on Ca²⁺ and K⁺ currents (30,31).

Reproducible evidence obtained in a total of more than 15,000 subjects shows that not only is QT interval duration modulated by NOS1AP, but that naturally occurring polymorphisms in this gene exert the strongest influence of any of the 10 loci now known to modulate QT in man (27). Very recently Kao et al (17) showed a positive association between some NOS1AP SNPs with incidence of sudden cardiac death in a large cohort of community SCD cases.

NOS1AP DNA Variants: From Population Studies to Long QT Syndrome

Given this robust evidence for the role of NOS1AP it became important to assess whether the same common variants account for the phenotypic variability among LQTS patients (6,32), a disease in which cardiac repolarization is profoundly affected by deleterious mutations. A recent study has suggested a role for NOS1AP in modulating LQTS phenotype in carriers of an A341V mutation in KCNQ1 gene (19). However it is unknown if the result also applies to carriers of different mutations and, most importantly, if NOS1AP SNPs may be used for risk stratification. One major challenge of such studies in LQTS is the difficulty of collecting a cohort of adequate size for which clinical data and DNA samples are available. With this objective in mind we queried the clinical database and DNA Biobank at the Maugeri Foundation and identified 901 patients in whom NOS1AP polymorphisms and changes in QTc and cardiac events could be studied. We chose to screen NOS1AP polymorphisms with a minor allele frequency higher than 10% among those that had been previously associated with QTc prolongation and/or increased risk of sudden death in different populations worldwide (8,17,26,27). We studied a population in the drug free-state in order to avoid the confusing effect of therapy on outcome, as a consequence our results are particularly pertinent to the role of NOS1AP SNPs on the natural history of LQTS.

NOS1AP rs4657139 and rs16847548 rare alleles were associated with a QTc prolongation of 7 and 8 ms respectively in this cohort. At first glance such an effect seems small, however it is within the range of the prolongation induced by drugs known to induce “acquired long QT syndrome” such as terfenadine or cisapride; drugs that have been removed from the market by regulatory authorities based on an excess mortality related to an average QT prolongation of 6 ms (33). As shown by studies performed in different databases in the last decades, QT interval duration is the most powerful predictor of risk of arrhythmic events in LQTS (3,4). Thus, NOS1AP can represent a meaningful modifier in this disease.

NOS1AP and Risk of Events in LQTS

To assess whether, besides prolonging the QT interval, NOS1AP SNPs contribute to modulation of susceptibility to cardiac events, we first used classification tree analysis that showed a significant stratification role for NOS1AP in patients with QTc<500 ms. Accordingly, Cox multivariate analysis showed that a QTc≧500 ms is the strongest predictor of events followed by gender while NOS1AP SNPs had a non-significant association in the entire 901 patient cohort.

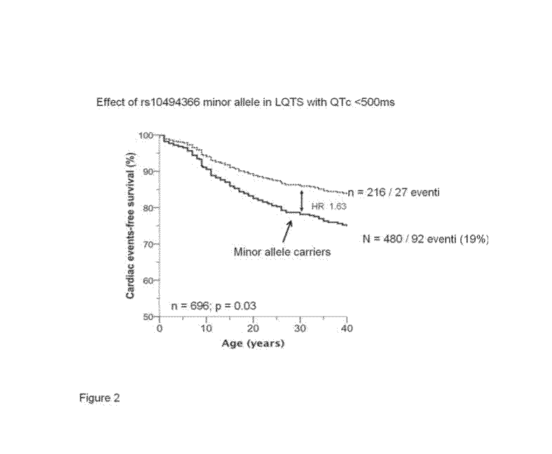

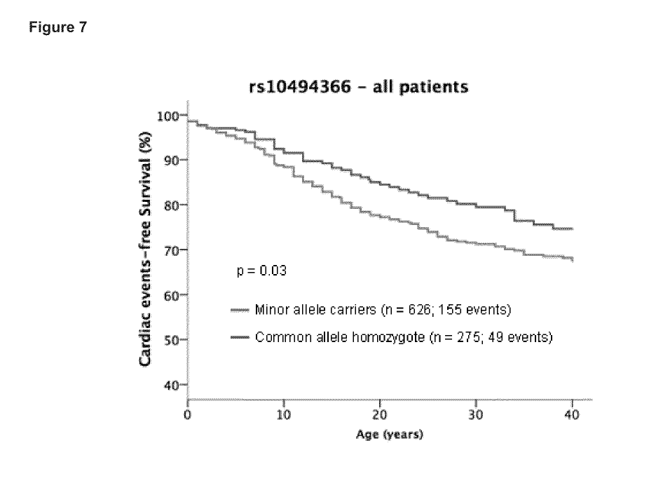

On the other hand in patients with QTc<500 ms (n=696) we found that the minor NOS1AP rs10494366 allele contributes quite significantly to risk stratification (HR 2.6, FIG. 2). Of note, not only the rate of cardiac events (syncope and cardiac arrest) but also the rate of cardiac arrests (hard end point) is significantly higher in the carriers of the minor allele (online supplement FIG. 7) although the total number of events to reach multivariate statistical significance.

The new finding that variants in the NOS1AP region signal potential for increased risk of cardiac events and cardiac arrest in patients with QTc<500 ms has significant epidemiological and clinical implications as this group of patients represent between 65-75% of the total population (3,34). The more pronounced effect in patients with QTc<500 msec is conceivable since the risk of events is already so high in patients with longer QTc that it is unlikely that the presence of a single SNP can substantially modulate the risk.

The observation here that NOS1AP rs10494366 minor allele has not been associated with risk of arrhythmias or sudden death in other investigations (17) is reminiscent of findings regarding positional variation in different individual NOS1AP SNP associations reported in prior studies on other populations (8,10,11,26,27,35). As suggested by Kao et al (17) genotyped SNPs may be considered proxy measures that are exposed to variable strength of the association with QT interval and SCD independently due to unequal linkage to the actual causal allelic variants. We therefore consider our finding on rs10494366 likely reflects population (36)—and/or disease-specific factors, as well as the possibility that there may be multiple functional elements within the NOS1AP region. This latter concept would also be consistent with the presence of markers of increased risk of arrhythmic events independent from NOS1AP effects on QTc (17).

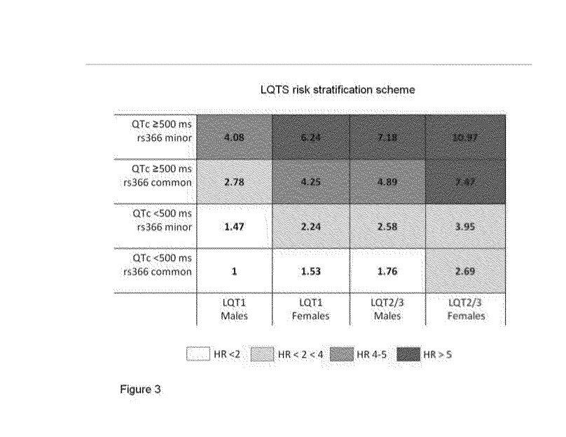

Based on the risk stratification model that we proposed in 2003 (4) which is now part of recommended practice guidelines for treatment of these patients (2), we analyzed our data to assess if consideration of NOS1AP variation would provide additional strength to that model. As shown in FIG. 3, genotyping variation in the NOS1AP gene has much potential to expand event risk stratification for many previously identified LQT1, LQT2 and LQT3 patients.

Practical Implications

When clinicians have to decide on therapy for LQTS patients, the dilemma of whether the patient may need an implantable defibrillator besides receiving beta blockers, should be based on risk stratification schemes derived from validated epidemiological studies. We previously showed (4) that QT interval, gender, and genetic locus are the most important determinants of risk in LQT1, LQT2 and LQT3. Now we provide evidence suggesting that NOS1AP SNP genotype may be a novel metric, which could help to refine risk stratification. This approach has particular potential to identify individuals at higher risk among those with QTc<500 msec: until now these patients, who represent the majority of LQTS clinical diagnoses, have been grouped in a lower risk category. It is thus possible that the introduction of the screening for NOS1AP SNPs may help the clinicians to identify subset of “QTc<500 ms patients” at higher risk, a supposition which will have to be validated by additional studies.

The potential of the discovery that NOS1AP variant genotypes may have validity towards this goal is illustrated in FIG. 3. Based on results of others, and our own (4) prior findings as extended here, we would consider that patients with a HR≧6 be further evaluated as potential high risk individuals who may benefit from ICD implantation. Patients with a HR between 4 and 6, depending on other clinical variables, may also be appropriate ICD candidates, whereas patients with a HR<4 in the absence of other risk factors (e.g. coronary or structural compromise) may be best treated with beta-blockers, again considering other cardiac risks and best clinical practices. Such considerations should obviously be in full compliance with clinical recommendations within the current AHA, ACC and ESC guidelines (2).

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

Methods

Study Population

From our clinical data base and DNA bank of LQTS patients (4-6) we identified Caucasian individuals with a clinical and/or molecular diagnosis of Romano Ward LQTS who had been screened for mutations in the KCNQ1, KCNH2, KCNE1, KCNE2 and SCN5A genes (4-6) and for whom we had DNA and clinical data available on natural history, cardiac events and response to therapy. Nine hundred and one affected patients entered the study: 520 probands and 381 family members (216 first-degree relatives from 153 families). Families with more than one mutation were excluded.

Local and national laws regarding biomedical research, personal data protection and confidentiality were respected. The study protocol was approved by the Maugeri Foundation Ethical Committee and all participants or their legal guardians signed an informed consent.

Definitions

QT interval was measured at the enrollment ECG. Heart rate QT correction was performed (QTc) in lead II (or lead I or III if it could not be measured in lead II) (4-6) from 12-lead ECGs with the use of Bazett's formula during stable heart rate (4-6). Based on previous studies (4,5) we used the QTc cut-off of ≧500 ms to define high-risk patients. Cardiac events were defined as syncope, torsade de pointes, cardiac arrest, or ICD shocks (4). As previously done in our studies (4-6) we used an observation period for cardiac events from birth to either attainment of age 40 or the initiation of anti-adrenergic therapy (beta blockers or left cardiac sympathetic denervation). Symptomatic patients are considered those who experienced a cardiac event according to the above definitions.

Based on a recent report (17) we genotyped three polymorphisms (rs4657139, rs16847548, rs10494366) located within the high linkage disequilibrium block in the 5′ region of the NOS1AP gene (8); three SNPs (rs4657139, rs16847548, rs10494366) showed MAF>0.10 and were included in this analysis. SNPs and haplotypes are indicated by an uppercase “A” to refer to the “major” (common) allele and a lower case “a” to refer to the “minor” (rare) allele. We have compared whether the dominant, additive or recessive model fitted better in order to predict QT or cardiac events. To do this, we have performed linear and logistic regression for QT and cardiac events, respectively, taking the SNP as the predictor variable in a dominant (2 categories—AA vs. (Aa or aa)), additive (3 categories—AA vs. Aa vs. aa and treated as continuous variable) or recessive (2 categories—AA or Aa vs. aa). After estimating the model, the model with highest likelihood was chosen: the largest R2 for linear model and the Nagelkerke's pseudo R2 for the logistic regression model. This analysis showed that the dominant model for QTc reached the best fit for both rs4657139 (p=0.004) and rs16847548 (p=0.008). For rs10494366 the linear trend was not significant for QTc (p=0.209), indicating no significant effect of rs10494366 allele variation on QT and either an additive or dominant model are equally, appropriate. A dominant model was the best fit for cardiac events (p=0.022). The SNPs having MAF>0.10 were included in the haplotypes in the following order: rs4657139 T>A, rs16847548 T>C and rs10494366 T>G. The D′ parameter and the frequency of haplotypes ≧0.10, defined by the unphased genotypes and a dominant model, were estimated with the R haplo.stats package. This program uses the expectation-maximization algorithm to estimate the probability of all and the most likely haplotype combinations. The inclusion cut off for haplotypes was set to an estimated probability ≧0.80.

Genetic Analysis

Genomic DNA was extracted from peripheral-blood lymphocytes according to standard procedures. Methods for mutation screening on LQTS genes have been reported in detail previously (4-6).

Multiplex-PCR amplicons were purified with exonuclease I-shrimp alkaline phosphatase (USB, Cleveland, Ohio) and used as a template for fluorescent dideoxy single-base extension of unlabeled oligonucleotide primers with the ABI PRISM SNaPshot™ Multiplex Kit (Applied Biosystems, Foster City, Calif.). A post-extension treatment with calf intestine alkaline phosphatase (New England BioLabs, Frankfurt, Germany), GeneScan™-120 LIZ™ internal size standard addition and heat denaturation, were carried out before sequencing in an ABI PRISM® 310 Genetic (Applied Biosystems).

Statistical Analysis

Analysis of variance, paired and unpaired t-tests and cross-tabulations with Fisher's exact test were used as appropriate. Classification tree analysis was used as an explorative unbiased technique to understand the structure of relationships between variables and to highlight cohort stratifications (20). We specifically used this approach in order to assess whether NOS1AP SNPs might add information to risk stratification in our LQTS population when analyzed together with the known metrics (QTc, gender and genotype). Classification trees were learned using the Orange data mining software (http://www.ailab.si/orange/) with gain ratio as purity measure. Correction of QTc vs. NOS1AP statistics for multiplicity of comparisons was performed by permutation test and permutation-corrected P-values were generated using an adaptive permutation approach as implemented in PLINK software. The cumulative probability of a first cardiac event before the age of 40 years and before antiadrenergic therapy was determined in the entire population and in each of the three most prevalent genetic loci with the use of the life-table method of Kaplan and Meier. Log-rank test with Bonferroni correction for multiplicity of comparisons was applied. Cox multivariate survivorship analyses were performed to evaluate the significance and independence of predictors of a first cardiac events or a first cardiac arrest alone. P values ≧0.05 were considered statistically significant. Unless otherwise specified, analyses were performed using SPSS software (v.16). P values <0.05 were considered statistically significant. Means are reported±standard deviation.

### EXAMPLE 2

The characteristics of the 901 LQTS patients (55% women, mean age at last follow up of 31±19 years) from 520 families included in the study are shown in Table 1. LQTS gene mutations were identified in seven hundred and seventy patients (85%): KCNQ1 (n=421; 55%), KCNH2 (n=243; 32%), SCN5A (n=83; 11%), KCNE1 (n=21; 2.7%) and KCNE2 (n=2; 0.3%). In the remaining 131 patients no mutations were found in the five genes. Two hundred four patients experienced cardiac events.

TABLE 1
Characteristics of Long QT-syndrome patients.
LQTS patients/probands 901/520

| Women | | 498 (55%) |
|---|---|---|
| Age at last follow up (years ± SD) | | 31 ± 19 |
| Symptomatic patients | | 204 (23%) |
| Cardiac arrest | | 26 (13%) |
| Syncope | | 178 (87%) |
| QTc (ms ± SD*) | | 476 ± 46 |
| LQT-locus (gene) | | 770 (85%) |
| LQT1 (KCNQ1) | | 421 (55%) |
| LQT2 (KCNH2) | | 243 (32%) |
| LQT3 (SCN5A) | | 83 (11%) |
| LQT5 (KCNE1) | | 21 (2.7%) |
| LQT6 (KCNE2) | | 2 (0.3%) |
| Not identified LQT-genotype | | 131 (15%) |
| NOS1AP SNPs MAF | rs4657139 T > A | 0.42 |
| | rs16847548 T > C | 0.29 |
| | rs10494366 T > G | 0.45 |
| Haplotype frequency† | AAA | 0.50 |
| | aaa | 0.26 |
| | aAa | 0.11 |

No significant differences in allelic frequencies in the subgroups have been identified.
*SD = Standard Deviation
†The order of the polymorphisms in the haplotype is rs4657139 T > A, rs16847548 T > C and rs10494366 T > G where uppercase letters represent the major alleles.

### EXAMPLE 3

Minor Allele Frequency (MAF) of SNPs and Haplotypes,

The MAF of the SNPs rs4657139 T>A, rs16847548 T>C and rs10494366 T>G MAF were 0.42, 0.29 and 0.45, respectively, and these were in linkage disequilibrium (D′>0.800). Out of the 901 subjects, NOS1AP haplotypes could be estimated with a probability ≧0.80 for 887 patients with 197 events (22.2%). The AAA, aaa, aAa haplotypes were the only ones presenting with >0.10 frequency (0.51, 0.26, and 0.11, respectively). SNP MAFs and common haplotype frequencies are shown in Table 1. Haplotype analysis provided no information beyond what was indicated by SNP analysis alone; thus these data are not included here. Detailed haplotype frequencies are reported in Table 2. In the following table it appears that only TTT, ACG and ATG have a frequency >0.1. Clearly the low frequency of cardiac events and the sample size (forcedly limited as compared with population study in normal individuals), do not permit the data to reach sufficient statistical power to analyze haplotypes with lower frequency in multivariate analysis.

TABLE 2

| rs4657139 | rs16847548 | rs10494366 | Freq. |
|---|---|---|---|
| T | T | T | 0.4950 |
| A | C | G | 0.2566 |
| A | T | G | 0.1143 |
| T | T | G | 0.0726 |
| A | T | T | 0.0239 |
| A | C | T | 0.0219 |
| T | C | G | 0.0093 |
| T | C | T | 0.0063 |

rs4657139 minor allele: “A”
rs16847548 minor allele: “C”
rs10494366 minor allele: “G”

### EXAMPLE 4

NOS1AP and Cardiac Repolarization

SNP analysis showed that carriers (Aa or aa) of minor alleles tagged by SNPs rs4657139 and rs16847548 had a longer QTc of 7 ms (p=0.047) and 8 ms (p=0.009) respectively, in this cohort, whereas there was no difference in QT associated with the minor allele of rs10494366 (Table 2).

TABLE 2
QTc duration in the overall LQTS patient
cohort shown by NOS1AP single SNPs.

| | AA | | a-carriers | | |
|---|---|---|---|---|---|
| SNP | QTc (ms) | n | QTc (ms) | n | p-value |
| rs4657139 | 472 ± 44 | 322 | 479 ± 46 | 579 | 0.047 |
| rs16847548 | 472 ± 45 | 455 | 480 ± 46 | 446 | 0.009 |
| rs10494366 | 475 ± 47 | 275 | 477 ± 45 | 626 | 0.486 |

### EXAMPLE 5

NOS1AP and Cardiac Events

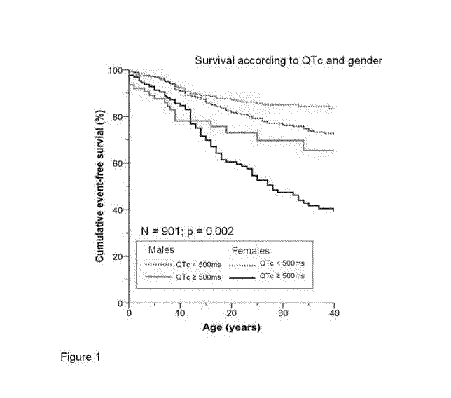

We assessed whether tag SNP variation in the 5′-NOS1AP gene were associated with an increased risk of cardiac events in the absence of anti-beta-adrenergic therapy. The percentage of symptomatic patients was higher among rs4657139 rare allele carriers as compared to AA homozygotes (25.2% vs. 18.0%; gender-adjusted HR=1.38, 1.01-1.86, 95% CI; p=0.040). Similarly, rs10494366 rare allele carriers were more symptomatic than AA homozygotes (24.8% vs. 17.8%; gender-adjusted HR=1.42, 1.03-1.96, 95% CI; p=0.031). Patients with the rs16847548 minor allele did not show an increase in risk of cardiac events. Similar univariate associations between SNPs and events were also observed with Kaplan Meier survival analysis (FIGS. 1-3 in the online supplement).

### EXAMPLE 6

QTc, Gender, and Genetic Locus in LQTS

Cohort records were analyzed over an observation period of 23±14 years, during which 204 patients (23%) experienced a cardiac event before age 40 or initiation of treatment with beta-blockers (Table 1). The mean age at first cardiac event was 14±10 years. Syncope occurred in 178/204 (87%) patients while cardiac arrest in the remaining 26 individuals (13%).

Among LQT1, LQT2 and LQT3 patients (n=747) Cox regression analysis showed that QTc, gender and genetic locus were significantly associated with clinical outcome as previously reported (4,21). Symptomatic patients had a longer QTc (500±48 ms n=156 vs. 466±40 ms n=591; p<0.0001; HR for QTc≧500 ms: 2.81, 95% CI: 2.02-3.90; p<0.0001) and were more likely to be female (68.5% females in the symptomatic group vs. 51.7% among asymptomatic cases; p<0.0001; HR for females=1.54, 95% CI: 1.02-2.16; p=0.014). Genetic locus was also associated with the outcome since LQT2 and LQT3 patients had more cardiac events as compared to LQT1cases (HR=1.67, 95% CI: 1.20-2.32; p<0.002).

### EXAMPLE 7

NOS1AP and Outcome Prediction in LQTS

We assessed the value of NOS1AP genotyping as a predictor of adverse outcomes and as a novel additional metric for risk stratification in LQTS. To this end we first performed classification tree analysis for unbiased identification of risk clusters. The results showed that NOS1AP genotype becomes informative regarding event risk only in patients with QTc<500 ms, while QTc and gender dominated the model for patients with QTc≧500 ms. Accordingly Cox regression in the cohort of 901 patients showed that none of the NOS1AP SNPs is a significant independent predictor of risk of events.

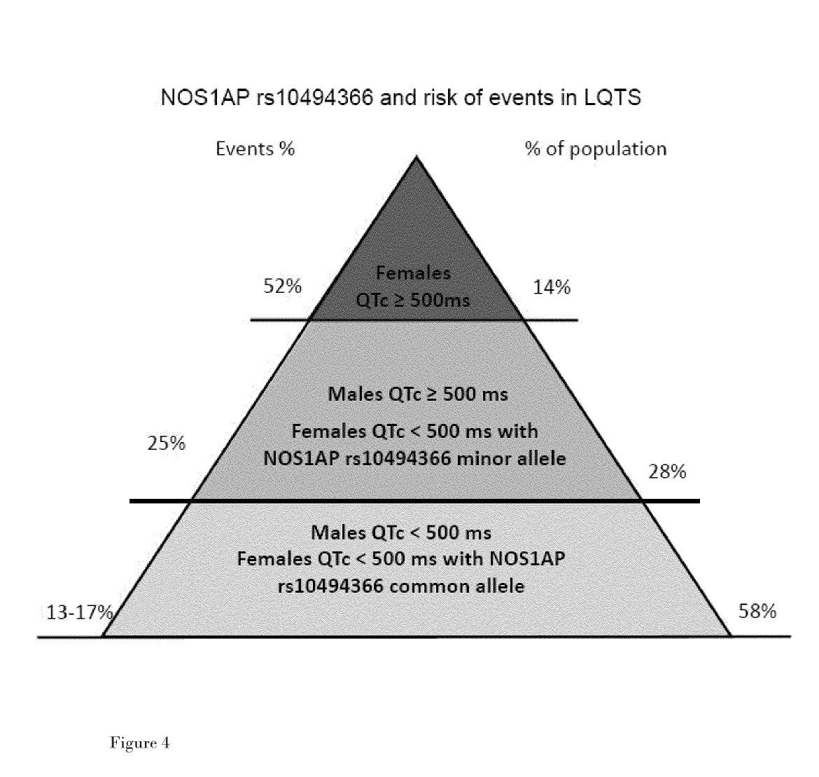

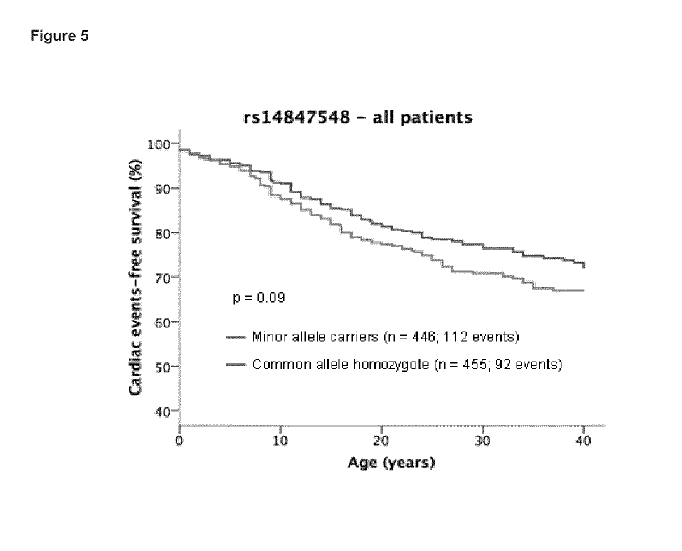

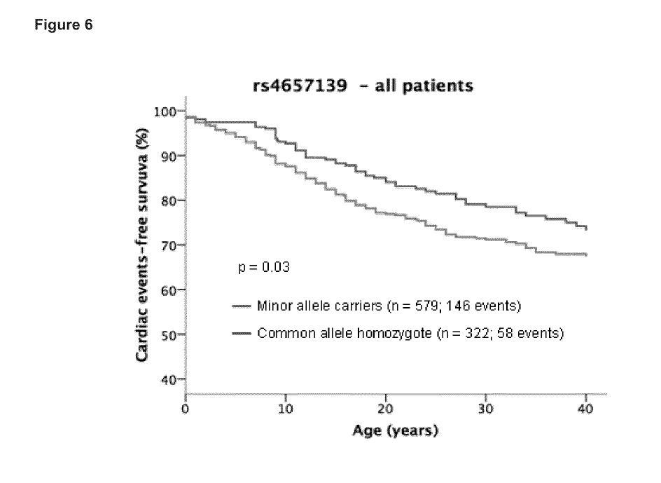

Overall, cardiac event distribution was remarkably different (p<0.002) in the four subgroups of patients when further stratified by QTc and gender: females with QTc≧500 ms were at highest risk of events (incidence 52% n=128), followed by a risk of 25% in males with QTc≧500 ms (n=77), a risk of 21% in females with QTc<500 ms (n=370), and a risk of 13% among males with QTc<500 ms (n=326). Kaplan Meier analyses showed significant differences of event rates in the four groups (FIG. 1 and online FIGS. 1-6).

When we focused on patients with QTc<500 ms, Cox regression analysis identified two independent markers of cardiac events: rs10494366 genotype (HR 1.63; 95% CI 1.06-2.50; p=0.03) (FIG. 2) and female gender (HR 1.61; 95% CI 1.10-2.35; p=0.01). Note that these data indicate female carriers of the minor allele of rs10494366 have a 2.6 fold increase in risk of arrhythmic events. Multiple regression analysis confirmed the lack of significant interaction between QTc and rs10994366. Interestingly when we restricted the analysis to cardiac arrest only there were 10 events in carriers of the rs10494366 minor allele and none in the common allele (log rank p=0.031; see online FIG. 7).

### EXAMPLE 8

NOS1AP and LQTS Mutation

Based on the published risk stratification schemes for LQT1, LQT2 and LQT3 patients (2,4) we investigated whether NOS1AP SNPs can add new insights for risk stratification in this group of patients. Univariate analysis showed that in the 747 patients genotyped as LQT1, LQT2 or LQT3, variant alleles in rs10494366 and rs4657139 are significantly associated with risk of cardiac events (log rank p=0.016 and p=0.037, respectively). Cox multivariate analysis demonstrated that: 1) the rs10494366 rare allele (HR 1.47; 95% CI 1.02-2.13; p=0.04): 2) LQT2 or LQT3 genotype (HR 1.76; 95% CI 1.25-2.48; p=0.001): 3) QTc≧500 ms (HR 2.78; 95% CI 1.99-3.85; p<0.0001), and 4) female gender (HR 1.53; 95% CI 1.09-2.16; p=0.01), were each significant predictors of increased risk of cardiac events (FIG. 3). No significant multivariate associations were observed with rs4657139 or rs16847548 (s4657139 rare allele: HR 1.27; 95% CI 0.89-1.80; p=0.18—rs16847548 rare allele: HR 1.10; 95% CI 0.89-1.51; p=0.58).

References

The disclosure of each reference cited is expressly incorporated herein.

### REFERENCES

1. Priori S G, Napolitano C, Schwartz P J, et al. Genetics of cardiac arrhythmias. Braunwald's Heart Disease. Philadelphia: Elsevier Saunders, 2004:689-695.
2. Zipes D P, Camm A J, Borggrefe M, et al. ACC/AHA/ESC 2006 guidelines for management of patients with ventricular arrhythmias and the prevention of sudden cardiac death: a report of the American College of Cardiology/American Heart Association Task Force and the European Society of Cardiology Committee for Practice Guidelines (Writing Committee to Develop Guidelines for Management of Patients With Ventricular Arrhythmias and the Prevention of Sudden Cardiac Death). J Am Coll Cardiol 2006; 48:e247-346.
3. Moss A J, Schwartz P J, Crampton R S, et al. The long QT syndrome. Prospective longitudinal study of 328 families. Circulation 1991; 84:1136-1144.
4. Priori S G, Schwartz P J, Napolitano C, et al. Risk stratification in the long-QT syndrome. N. Engl. J Med 2003; 348:1866-1874.
5. Priori S G, Napolitano C, Schwartz P J, et al. Association of long QT syndrome loci and cardiac events among patients treated with beta-blockers. JAMA: The Journal of the American Medical Association 2004; 292:1341-1344.
6. Napolitano C, Priori S G, Schwartz P J, et al. Genetic testing in the long QT syndrome: development and validation of an efficient approach to genotyping in clinical practice. JAMA. 2005; 294:2975-2980.
7. Bezzina C R, Verkerk A O, Busjahn A, et al. A common polymorphism in KCNH2 (HERG) hastens cardiac repolarization. Cardiovasc. Res 2003; 59:27-36.
8. Arking D E, Pfeufer A, Post W, et al. A common genetic variant in the NOS1 regulator NOS1AP modulates cardiac repolarization. Nat. Genet. 2006; 38:644-651.
9. Newton-Cheh C, Guo C Y, Wang T J, O'Donnell C J, Levy D, Larson M G. Genome-wide association study of electrocardiographic and heart rate variability traits: the Framingham Heart Study. BMC. Med. Genet. 2007; 8 Suppl 1:S7.
10. Aarnoudse A J, Newton-Cheh C, de Bakker P I, et al. Common NOS1AP variants are associated with a prolonged QTc interval in the Rotterdam Study. Circulation 2007; 116:10-16.
11. Post W, Shen H, Damcott C, et al. Associations between genetic variants in the NOS1AP (CAPON) gene and cardiac repolarization in the old order Amish. Hum. Hered. 2007; 64:214-219.
12. Lehtinen A B, Newton-Cheh C, Ziegler J T, et al. Association of NOS1AP genetic variants with QT interval duration in families from the Diabetes Heart Study. Diabetes 2008; 57:1108-1114.
13. Tobin M D, Kahonen M, Braund P, et al. Gender and effects of a common genetic variant in the NOS1 regulator NOS1AP on cardiac repolarization in 3761 individuals from two independent populations. Int. J. Epidemiol. 2008; 37:1132-1141.
14. Raitakari O T, Blom-Nyholm J, Koskinen T A, Kahonen M, Viikari J S, Lehtimaki T. Common variation in NOS1AP and KCNH2 genes and QT interval duration in young adults. The Cardiovascular Risk in Young Finns Study. Ann. Med. 2008: 1-8.
15. Marjamaa A, Newton-Cheh C, Porthan K, et al. Common candidate gene variants are associated with QT interval duration in the general population. J. Intern. Med. 2008.
16. Eijgelsheim M, Aarnoudse A L, Rivadeneira F, et al. Identification of a common variant at the NOS1AP locus strongly associated to QT-interval duration. Hum Mol Genet 2009; 18:347-57.
17. Kao W H, Arking D E, Post W, et al. Genetic variations in nitric oxide synthase 1 adaptor protein are associated with sudden cardiac death in US white community-based populations. Circulation 2009; 119:940-951.
18. Spooner P M. Sudden Cardiac Death: The Larger Problem . . . the Larger Genome. J Cardiovasc Electrophysiol 2009; 20:585-596.
19. Schwartz P J, M.C. M, Insolia R, et al. NOS1AP is a genetic modifier of the long-QT syndrome. Circulation, 2009; 120:1657-1663.
20. Breiman L F, J. H. Olshen, R. A. Stone, J. C. Classification and Regression Trees. New York: Chapman & Hall, 1994.
21. Zareba W, Moss A J, Schwartz P J, et al. Influence of genotype on the clinical course of the long-QT syndrome. International Long-QT Syndrome Registry Research Group. N. Engl. J. Med. 1998; 339:960-965.
22. Sesti F, Abbott G W, Wei J, et al. A common polymorphism associated with antibiotic-induced cardiac arrhythmia. Proc. Natl. Acad. Sci U.SA 2000; 97:10613-10618.
23. Yang P, Kanki H, Drolet B, et al. Allelic variants in long-QT disease genes in patients with drug-associated torsades de pointes. Circulation 2002; 105:1943-1948.
24. Pfeufer A, Jalilzadeh S, Perz S, et al. Common variants in myocardial ion channel genes modify the QT interval in the general population: results from the KORA study. Circ. Res. 2005; 96:693-701.
25. Gouas L, Nicaud V, Berthet M, et al. Association of KCNQ1, KCNE1, KCNH2 and SCN5A polymorphisms with QTc interval length in a healthy population. Eur. J Hum. Genet. 2005; 13:1213-1222.
26. Newton-Cheh C, Eijgelsheim M, Rice K M, et al. Common variants at ten loci influence QT interval duration in the QTGEN Study. Nat Genet. 2009; 41:399-406.
27. Pfeufer A, Sanna S, Arking D E, et al. Common variants at ten loci modulate the QT interval duration in the QTSCD Study. Nat Genet. 2009; 41:407-414.
28. Suzuki N, Motohashi N, Uezumi A, et al. NO production results in suspension-induced muscle atrophy through dislocation of neuronal NOS. J Clin Invest 2007; 117:2468-2476.
29. Eastwood S L. Does the CAPON gene confer susceptibility to schizophrenia? PLoS. Med. 2005; 2:e348.
30. Chang K C, Barth A S, Sasano T, et al. CAPON modulates cardiac repolarization via neuronal nitric oxide synthase signaling in the heart. Proc. Natl. Acad. Sci. U.S.A 2008; 105:4477-4482.
31. Jaffrey S R, Snowman A M, Eliasson M J, Cohen N A, Snyder S H. CAPON: a protein associated with neuronal nitric oxide synthase that regulates its interactions with PSD95. Neuron 1998; 20:115-124.
32. Vincent G M, Timothy K W, Leppert M, Keating M. The spectrum of symptoms and QT intervals in carriers of the gene for the long-QT syndrome. N. Engl. J Med. 1992; 327:846-852.
33. Pratt C M, Ruberg S, Morganroth J, et al. Dose-response relation between terfenadine (Seldane) and the QTc interval on the scalar electrocardiogram: Distinguishing a drug effect from spontaneous variability. American Heart Journal 1996; 131:472-480.
34. Locati E H, Zareba W, Moss A J, et al. Age- and sex-related differences in clinical manifestations in patients with congenital long-QT syndrome: findings from the International LQTS Registry. Circulation 1998; 97:2237-2244.
35. Eijgelsheim M, Newton-Cheh C, Aarnoudse A L, et al. Genetic variation in NOS1AP is associated with sudden cardiac death: evidence from the Rotterdam Study. Hum Mol Genet 2009.
36. Novembre J, Johnson T, Bryc K, et al. Genes mirror geography within Europe. Nature 2008; 456:98-101.

1. A method of stratifying female patients for risk of adverse cardiac events, syncope, Torsade de pointes, or cardiac arrest, wherein the patients have a syndrome selected from the group consisting of LQTS1-10, short QT syndrome, Brugada Syndrome, catecholaminergic polymorphic ventricular tachycardia (CPTV) and cryptic, familial QT abnormality, but wherein the patients do not have a KCNQ1:A341V mutation, comprising the steps of:
testing a female patient's genomic sequences for the presence of a minor allele of NOS1AP identifiable with a tag SNP selected from the group consisting of rs10494366, rs54657139, rs16847548, and markers in positive linkage disequilibrium with said minor allele;
testing to determine electrographic (QT) interval in the female patient;
if the patient has (a) said minor allele or said markers in positive linkage disequilibrium, and (b) a QT prolongation, then identifying the patient as having a higher risk than the average for a population of females with said syndrome who do not have both a QT prolongation and said minor allele.

2. A method of stratifying male patients for risk of adverse cardiac events, syncope, Torsade de pointes, or cardiac arrest, wherein the patients have a syndrome selected from the group consisting of LQTS1-10, short QT syndrome, Brugada Syndrome, catecholaminergic polymorphic ventricular tachycardia (CPTV) and cryptic, familial QT abnormality, but wherein the patients do not have a KCNQ1:A341V mutation, comprising the steps of:
testing in a male patient's genomic sequences for the presence of a minor allele of NOS1AP identifiable with a tag SNP selected from the group consisting of rs10494366, rs54657139, rs16847548, and a marker in positive linkage disequilibrium with said minor allele;
identifying the patient as having a higher risk than the average for a population of male patients that have said syndrome if the patient has said minor allele or a marker in positive linkage disequilibrium.

3. The method of claim 1 further comprising the step of: placing the patient in a clinical trial group comprising other females having said minor allele and a QT prolongation.

4. The method of claim 2 further comprising the step of: placing the patient in a clinical trial group comprising other males having said minor allele.

5. The method of claim 1 wherein the patient is identified as having a higher risk if the patient has a QTc (QT corrected for heart rate) of less than 500 msec

6. The method of claim 1 or 2 further comprising the step of: prescribing anti-beta adrenergic drugs to the patient if identified as being at higher risk.

7. The method of claim 1 or 2 further comprising: prescribing implantation of an implantable cardio-defibrillator (ICD) in the patient, if the patient is identified as being at higher risk.

8. The method of claim 1 or 2 further comprising: implanting an implantable cardio-defibrillator (ICD) in the patient, if the patient is identified as being at higher risk.

9. The method of claim 1 or 2 wherein the minor allele or the marker in positive linkage disequilibrium with at least one of said minor alleles is determined by hybridization of patient genomic sequences to a nucleic acid probe.

10. The method of claim 9 wherein nucleic acid probe is in an array of probes.

11. The method of claim 9 wherein the nucleic acid probe is immobilized on a solid support.

12. The method of claim 1 or 2 wherein the minor allele or the marker in positive linkage disequilibrium with at least one of said minor alleles is determined by nucleic acid sequence determination.

13. The method of claim 1 or 2 wherein the minor allele is determined by testing for and determining presence of a haplotype associated with at least one of said minor alleles or a marker in positive linkage disequilibrium with said minor allele.

14. A method of stratifying female patients for risk of adverse cardiac events, syncope, Torsade de pointes, or cardiac arrest, wherein the patients have a prolonged QT interval and wherein the patients have a syndrome selected from the group consisting of LQTS1-10, short QT syndrome, Brugada Syndrome, catecholaminergic polymorphic ventricular tachycardia (CPTV) and cryptic, familial QT abnormality, comprising the steps of:
determining in genomic sequences of one of the patients a minor allele of NOS1AP at rs10494366, rs54657139, rs16847548, or a marker in linkage disequilibrium with the minor allele;
identifying the patient as having a higher risk than the average for a female population having prolonged QT and said syndrome.

15. The method of claim 14 wherein the minor allele or the marker in positive linkage disequilibrium with at least one of said minor alleles is determined by nucleic acid sequence determination.

16. The method of claim 14 wherein the patients have a QTc (QT corrected for heart rate) of less than 500 msec

17. A method of stratifying female patients for risk of adverse cardiac events, syncope, Torsade de pointes, or cardiac arrest, wherein genomic sequences of the patients have a minor allele of NOS1AP identifiable with a tag SNP rs10494366, rs54657139, rs16847548, or a marker in linkage disequilibrium with rs10494366, rs54657139, rs16847548, and wherein the patients have a syndrome selected from the group consisting of LQTS1-10, short QT syndrome, Brugada Syndrome, catecholaminergic polymorphic ventricular tachycardia (CPTV) and cryptic, familial QT abnormality, comprising the steps of:
determining a prolonged QT interval in one of the female patients;
identifying the patient as having a higher risk than the average for a population having the minor allele of NOS1AP at rs10494366, rs54657139, rs16847548, or a marker in linkage disequilibrium with the minor allele.

18. The method of claim 17 wherein the patients are identified as having a higher risk if they are determined to have a QTc (QT corrected for heart rate) of less than 500 msec

19. A method of stratifying patients for risk of adverse cardiac events and cardiac arrest, comprising the steps of:
determining in a patient's genomic sequences a minor allele of NOS1AP at rs54657139, rs16847548, or a marker in linkage disequilibrium with rs54657139, rs16847548, wherein the patient is taking or may be prescribed a drug that prolongs QT;
identifying the patient as having a higher risk of adverse cardiac events and cardiac arrest than the average for a population taking the drug that does not have said minor allele.

20. The method of claim 19 wherein the minor allele or the marker in positive linkage disequilibrium with said minor allele is determined by nucleic acid sequence determination.
